SEQUENCE LISTING (8 sequences)

SEQ ID NO: 1 - DNA, 23 bp, homo sapien
tgttgctgggagctgttctactg

SEQ ID NO: 2 - DNA, 23 bp, homo sapien
atgtctcccttaggaccaataag

SEQ ID NO: 3 - DNA, 20 bp, homo sapien
ccatcctgcgtctggacctg

SEQ ID NO: 4 - DNA, 20 bp, homo sapien
gtaacagtccgcctagaagc

SEQ ID NO: 5 - DNA, 21 bp, homo sapien
gcaaatccttgctgttccaat

SEQ ID NO: 6 - DNA, 21 bp, homo sapien
ggagaaggcttcccagctttt

SEQ ID NO: 7 - DNA, 23 bp, homo sapien
cccagagtcatgagtcgaaggag

SEQ ID NO: 8 - DNA, 22 bp, homo sapien
caggcgcatgagtacttctcgg

(19) United States
(12) Patent Application Publication
(10) Pub. No.: US 2002/0132773 A1
(43) Pub. Date: Sep. 19, 2002

(54) Methods for reducing fat by administration of adiponectin

(75) Inventors: Paul W. Kincade, Nichols Hill, OK (US); Takafumi Yokuta, Norman, OK (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Oklahoma Medical Research Foundation

(21) Appl. No.: 10/099,355

(22) Filed: Mar. 14, 2002

Related U.S. Application Data
(60) Provisional application No. 60/275,755, filed on Mar. 14, 2001.

(51) Int. Cl. A61K 38/17

(52) U.S. Cl. 514/12

(57) ABSTRACT

The stromal cells that support blood cell production within bone marrow are pre-adipocytes and functional interactions with marrow fat cells have long been suspected. Adiponectin was recently isolated as an adipocyte product and shown to have structural similarities to Clq as well as members of the TNF superfamily. It suppresses myeloid differentiation in short term bone marrow cultures and also inhibits macrophage functions. These observations raised the possibility that precursors of other blood cell lineages interact with fat cells in marrow via adiponectin. It has now been determined that the factor blocks B lymphopoiesis in Whitlock-Witte type bone marrow cultures, but not the production of myeloid cells in Dexter cultures. Several observations suggest that non-lymphoid cells represent the target of this new mediator, and the B lymphoid lineage is only indirectly influenced. Highly purified lymphocyte precursors in stromal cell-free, serum-free cultures were unaffected by adiponectin. Similarly, there was no influence on IL-7 responding pro-B cells in clonal assays. However, the cytokine dramatically inhibited adipogenesis in culture, suggesting that it may normally be a feedback inhibitor of this process. PCR analyses revealed that COX-2 is induced on exposure of cloned preadipocytes to adiponectin, resulting in prostaglandin release. This is critical to the inhibition of adipogenesis, because a COX-2 inhibitor, DUP-697 blocked the response of preadipocytes to adiponectin. Furthermore, fat cell formation in response to adiponectin was defective in mice with disruption of the COX-2 gene. In contrast, expression of TNF-α, TGF-β, interferons and a new interferon-like cytokine known as limitin are not up-regulated by adiponectin. These results also demonstrate that COX-2 inducers can be used to cause weight loss, and that COX-2 inhibitors will cause weight gain.

This application claims prioity to U.S. S. No. 60/275,755 filed Mar. 14, 2001.

The United States government has rights in this application by virtue of grants AI 45864, AI 33085, and AI 20069 from the National Institutes of Health to Paul Kincade.

### BACKGROUND OF THE INVENTION

The present invention is generally in the field of causing weight loss, specifically by administration of adiponectin.

The prevalence of obesity has reached epidemic proportions in most developed countries and carries with it staggering mortality and morbidity statistics. Obesity is a well established risk factor for a number of potentially life-threatening diseases such as atherosclerosis, hypertension, diabetes, stroke, pulmonary embolism, and cancer. (Meisler J., St. Jeor S. 1996. Am J Clin Nutr. 63:409S-411S; Bray G. 1996. Endocrin Metab Clin North Aimer. 25:907-919). Furthermore, it complicates numerous chronic conditions such as respiratory diseases, osteoarthritis, osteoporosis, gall bladder disease, and dyslipidemias. The enormity of this problem is best reflected in the fact that death rates escalate with increasing body weight. More than 50% of all-cause mortality is attributable to obesity-related conditions once the body mass index (BMI) exceeds 30 kg/m², as seen in 35 million Americans. (Lee L., Paffenbarger R. 1992. JAMA. 268:2045-2049). By contributing to greater than 300,000 deaths per year, obesity ranks second only to tobacco smoking as the most common cause of potentially preventable death. (McGinnis J., Foege W. 1993. MA.270:2207-2212).

Accompanying the devastating medical consequences of this problem is the severe financial burden placed on the health care system in the United States. The estimated economic impact of obesity and its associated illnesses from medical expenses and loss of income are reported to be in excess of $68 billion/year. (Colditz G. 1992. Am J Clin Nutr. 55:503S-507S; Wolf A., Colditz G. 1996. Am J. Clin Nutr. 63:466S-469S; Wolf A., Colditz G. 1994. Pharmacoeconomics. 5:34-37). This does not include the Heater than $30 billion per year spent on weight loss foods, products, and programs. (Wolf A., Colditz G. 1994. Pharmacoeconomics. 5:34-37; Ezzati, et al.1992. Vital health Stat [2]. 113).

In 1990, the US government responded to the crisis by establishing as a major national health goal the reduction in the prevalence cf obesity to (20% of the population by the year 2000. (Public Health Service. Healthy people 2000: national health promotion and disease prevention objective. 1990; US Department of Health and Human Services Publication PHS 90-50212). In spite of this objective, the prevalence of overweight people in the United States has steadily increased, reaching an astounding 33.0% in the most recent National Health and Nutrition Examination Survey (1988-1991). (Kuczmarski, et al. 1994. JAMA. 272:205-211). Furthermore, the mean BMI has also increased over this period by 0.9 kg/m². This alarming trend has not occurred as the result of lack of effort. On the contrary, an estimated 25% of men, 50% of women, and 44% of adolescents are trying to lose weight at any given time. (Robinson, et al. J Amer Diabetic Assoc. 93:445-449). Rather, the 31% increase in rate and 8% increase in overweight prevalence over the past decade is a testimony of the fact that obesity is notoriously resistant to current interventions. (NIH Technology Assessment Conference Panel. 1993. Ann Intern Med. 119:764-770).

A major reason for the long-term failure of established approaches is their basis on misconceptions and a poor understanding of the mechanisms of obesity. Conventional wisdom maintained that obesity is a self-inflicted disease of gluttony. Comprehensive treatment programs, therefore, focused on behavior modifications to reduce caloric intake and increase physical activity using a myriad of systems. These methods have limited efficacy and are associated with recidivism rates exceeding 95%.

Failure of short-term approaches, together with the recent progress made in elucidating the pathophysiology of obesity, have lead to a reappraisal of pharmacotherapy as a potential long-term, adjuvant treatment. (National Task Force on Obesity. 1996. JAMA. 276:1907-1915; Ryan, D. 1996. Endo Metab Clin N Amer. 25:989-1004). The premise is that body weight is a physiologically controlled parameter similar to blood pressure, and obesity is a chronic disease similar to hypertension. The goal of long-term (perhaps life-long) medical therapy would be to facilitate both weight loss and subsequent weight maintenance in conjunction with a healthy diet and exercise. To assess this approach, the long-term efficacy of currently available drugs must be judged against that of non-pharmacological interventions alone. The latter approach yields an average weight loss of 8.5 kg at 21 weeks of treatment and only maintains 50% of the weight reduction at 4 years in 10-30% of the patients. (Wadden T. 1993. Ann Intern Med. 119:688-693; Kramer, et al.1989. Int J Obes. 13:123-136). The few studies that have evaluated long-term (greater than 6 months) single-drug (Guy-Gran, et al. 1989. Lancet. 2:1142-1144; Goldstein, et al. 1994 Int J Obes. 18:129-135; Goldstein, et al. 1993. Obes Res. 2:92-98) or combination therapy (Weintraub M. 1992. Clin Pharmacol. Ther. 51:581-585) show modest efficacy compared with placebo in the reduction of body weight.

Fat metabolism is complicated. Multiple functions, attributed to adipose tissue include thermoregulation, energy storage, estrogen synthesis and cytokine production. While fat cells and their precursors have been the focus of many studies involving obesity, they also constitute a normal component of bone marrow. Indeed, adipocytes, hematopoiesis-supporting stromal cells, osteoblasts and myocytes appear to derive from common mesenchynmal stem cells in that tissue. Cloned preadipocyte lines with the potential for differentiation in culture have been extremely valuable for understanding the molecular regulation of differentiation. Agents that induce fat cell formation from these precursors include insulin, hydrocortisone, methylisobutylxanthine (MIBX) and ligands for peroxisome proliferator activator receptors (PPAR). On the other hand, many findings indicate that adipogenesis is also controlled through negative feedback mechanisms. For example, adipose tissue produces leptin, plasminogen activator inhibitor type 1 (PAI-1), tumor necrosis factor alpha (TNF-α), transforming growth factor type beta (TGF-β), and prostaglmdin E₂ (PGE₂); agents that are thought to block fat cell formation.

Fat cells are conspicuous in normal bone marrow and have long been suspected to have an influence on hematopoiesis. Indeed, adipogenesis alters expression of extracellular matrix and cytokines in bone marrow, affecting hematopoiesis both directly and indirectlly Preadipocytes support blood cell formation in culture and fully differentiated fat cells produce less CSF-1 than their precursors. Expression of stem cell factor, interleukin-6 and leukemia inhibitory factor as well as hematopoiesis-supportive activity declined with terminal adipocyte differentiation of an embryo derived stromal line. The fat cell product, leptin, promotes osteoblast formation and hematopoiesis, while inhibiting adipogenesis.

All medications currently used to treat or prevent obesity are directed at the adipocyte compartment of the tissue and work by either decreasing energy availability or increasing energy output. These agents can be placed into three categories based on mechanism. (National Task Force on Obesity. 1996. JAMA. 276:1907-1915).

Reduction of Energy Intake

This approach is directed at reducing food intake by decreasing appetite or increasing satiety. These ‘anorexiant’ drugs affect neurotransmitter activity by acting on either the catecholaminergic system (amphetamines, benzphetamine, phendimetrazine, phentermine, mazindol, diethylpropion, and phenylpropanolamine) or the serotonergic system (fenfluramine, dexfenfluramine, fluoxetine, sertraline, and other antidepressant selective serotonin reuptake inhibitors [SSRI]).

Reduction in Absorption of Nutrients

Drugs in this category block the action of digestive enzymes or absorption of nutrients. An (example of this type of drug is orlistat, which inhibits gastric and pancreatic lipase activity. (Drent M., van der Veen E. 1995. Obes Res. 3(suppl 4):623S-625S). These medications are experimental in the United States and not available for he treatment of obesity.

Increase in Energy Expenditure

An increase in energy expenditure may be accomplished by increasing metabolic rate, for example, through changes in sympathetic nervous system tone or uncoupling of oxidative phosphorylation. Drugs that affect thermogenesis-metabolism include ephedrine alone or in combination with caffeine and/or aspirin, (Passquali R., Casimirri F. 1993 Int J Obes. 17(suppl 1):S65-S68) and BRL 26830A, an adrenoceptor agonist. (Connacher, et al.1992. Am J Clin Nutr. 55:258S-261 S). This class of medications is not approved by the FDA for weight control.

Currently, no single drug regimen emerges as superior in either promoting or sustaining weight loss. Surgical intervention,;, such as gastric partitioning procedures, jejunoileal bypass, and vagotomy, have also been developed to treat severe obesity. (Greenway F. 1996. Endo Metab Clin N Amer. 25:1005-1027). Although advantageous in the long run, the acute risk benefit ratio has reserved these invasive procedures for morbidly obese patients according to the NIH consensus conference on obesity surgery (BMI greater than 40 kg/m²). (NIH Conference. 1991. Ann Intern Med. 115:956-961). Therefore, this is not an alternative for the majority of overweight patients, unless and until they become profoundly obese and are suffering the attendant complications.

There is no medical or surgical treatment for obesity that is directed at the vascular compartment of the tissue.

It is therefore an object of the present invention to provide an alternative treatment to reduce obesity.

### SUMMARY OF THE INVENTION

Adiponectin, recently isolated as an adipocyte product and shown to have structural similarities to Clq, as well as to members of the tumor necrosis factor (TNF) superfamily, suppresses myeloid differentiation in short term bone marrow cultures and also inhibits macrophage functions. Adiponectin dramatically inhibits adipogenesis in culture, suggesting that it may normally be a feedback inhibitor of this process. PCR analyses revealed that COX-2 is induced on exposure of cloned pre-adipocytes to adiponectin, resulting in prostaglandin release. This is critical to the inhibition of adipogenesis, because a COX-2 inhibitor, DUP-697 blocked the response of preadipocytes to adiponectin. Furthermore, fat cell formation in response to adiponectin was defective in mice with disruption of the COX-2 gene. In contrast, expression of TNF-α, TGF-β, interferons and a new interferon-like cytokine known as limitin are not up-regulated by adiponectin. It has now been shown that adiponectin is present within normal bone marrow and can inhibit fat cell formation by marrow derived stromal cells through a COX-2 dependent mechanism. These findings suggest a new mechanism for regulation of preadipocyte differentiation and possible roles for fat in hematopoietic tissue.

These results support the use of adiponectin to decrease fat in adipocytes and associated fatty tissue. The adiponectin, as the 32 KD protein or a trimer thereof, or functionally equivalent fragments thereof, can be administered using methods known to those skilled in the art to achieve a decrease in fat.

### DETAILED DESCRIPTION OF THE INVENTION

I. Adiponectin Formulations

Adiponectin is an adipocyte-specific secretory protein and a new member of the family of soluble defense collagens, in hematopoiesis and immune responses. Adiponectin is a plasma protein secreted exclusively from adipocytes. In plasma from healthy humans, it exists in concentrations ranging from 1.9 to 17.0 μg/mL. Four groups independently discovered this protein designated Acrp30, adipoQ, or adiponectin that represents a major fat cell-restricted product in mouse and man (Scherer,et al., J. Biol. Chem. 270:26746-26749 (1995); Hu, et al., (1996) J. Biol. Chem. 271:10697-10703; Maeda, et al., Biochem. Biophys. Res. Commun. 221:286-289 (1996); Nakano, et al., J. Biochem. (Tokyo). 120:803-812 (1996)). It was also isolated from human serum and termed GBP28. The production of adiponectin increases in accordance with the differentiation of preadipocytes to adipocytes and is inhibited by TNF-α). Adipocytes utilize a specialized secretory compartment to release this protein (Bogan, J. S., and Lodish, H. F. J. Cell Biol. 146:609-620 (1999)).

Adiponectin suppresses colony formation from colony-forming unit (CFU)-granulocyte-macrophage, CFU-macrophage, and CFU-granulocyte, but has no effect on that of burst-forming units—erythroid or mixed erythroid-myeloid CFU. Adiponectin also inhibits proliferation of 4 of 9 myeloid cell lines, but does not suppress proliferation of erythroid or lymphoid cell lines except for one cell line. These results suggest that adiponectin predominantly inhibits proliferation of myelomonocytic lineage cells. At least one mechanism of the growth inhibition is induction of apoptosis because treatment of acute myelomonocytic leukemia lines with adiponectin induces the appearance of aubdipold peaks and oligonucleosomal DNA fragmentation. Aside from inhibiting growth of myelomonocytic progenitors, adiponectin suppresses mature macrophage functions. Treatment of cultured macrophages with adiponectin significantly inhibits their phagocytic activity and their lipopolysaccharide-induced production of tumor necrosis factor-α. Suppression of phagocytosis by adiponectin is mediated by one of the complement Clq receptors, ClqRp, because this function is completely abrogated by the addition of an anti-ClqRp monoclonal antibody (Yokota, Blood. 96:1.723-1732 (2000). These observations suggest that adiponectin is an important negative regulator in hematopoiesis and immune systems and that it is involved in ending inflammatory response through its inhibitory functions.

Adiponectin is composed of 244 amino acid residues containing a short noncollagenous N-terminal segment followed by a collagen-like sequence. Maeda, et al. J. Biochem. Biophys. Res. Commun. 221 (2), 286-289 (1996) MEDLINE 96224171. Adiponectin is a homotrimer that is similar in size and overall structure to complement protein Clq, with particularly high homology in the C-terminal globular domain. The crystal structure of adiponectin revealed additional high similarity between the same domain and TNF-α). These structural features suggest that adiponectin belongs to a family of proteins identified as soluble defense collagens and including complement Clq and the collections mannose-binding lectin (MBL), lung surfactant protein A (SP-A), lung surfactant protein D, and conglutinin. The collectins play important roles in the innate humoral immune system. These proteins can identify foreign pathogens by detecting specific carbohydrate structures uniquely present on microorganisms, and they subsequently interact with phagocytic cells or the complement system to bring about killing and clearance of targets without involvement of antibodies. Lack or low levels of collectin expression cause increased susceptibility to infections, especially in infants, whose specific immune systems for various pathogens have not fully developed.

Adiponectin has applications in diabetes and obesity because of its influence on glucose and lipid metabolism. As described below, it has been found that brown fat in normal human bone marrow contains this protein. Recombinant adiponectin blocked fat cell formation in long-term bone marrow cultures and inhibited the differentiation of cloned stromal preadipocytes. Adiponectin also caused elevated expression of cyclooxygenase-2 by these stromal cells and induced release of prostaglandin E₂. The cyclooxygenase-2 inhibitor Dup-697 prevented the inhibitory action of adiponectin on preadipocyte differentiation, suggesting involvement of stromal cell derived prostenoids. Furthermore, adiponectin failed to block fat cell generation when bone marrow cells were derived from B6,₁₂₉S-^{Ptgs2tmlJed} (Cyclooxygenase-2^{+/-}) mice. These observations show that preadipocytes represent direct targets for adiponectin action, establishing a paracrine negative feedback loop for fat regulation. They also link adiponectin to the cyclooxygenase-2 dependent prostaglandins that are critical in this process.

Normal biological activities of adiponectin are poorly understood, but findings suggest potential involvement in obesity, cardiovascular disease, and diabetes. Production and circulating protein concentrations are suppressed in obese mice and humans (Hu, et al., J. Biol. Chem. 271:10697-107032 (1996); Arita, et al. Biochem. Biophys. Res. Commun. 257:79-83 (1999)). Low plasma levels may be a risk factor in coronary heart disease and concentrations are also significantly reduced in type 2 diabetes (Ouchi, et al., Circulation. 100:2473-2476 (1999); Hotta, et al., Diabetes. 50:1126-1133 (2001)). The ability of adiponectin to lower glucose and reverse insulin resistance suggests that it may 30 have application as a diabetes drug (Yamauchi, et al., Nat. Med. 7:941-946 (2001); Berg, et al. Nat. Med. 7:947-953 (2001)). Furthermore, a proteolytically cleaved fragment of adiponectin was shown to cause weight loss in obese animals (Fruebis, et al., Proc. Natl. Acad. Sci. USA. 98:20(15-2010 (2001)). This protein directly or indirectly affects at least four cell types. Adiponectin modulates NF-κB mediated signals in human aortic endothelial cells, presumably accounting for their reduced adhesiveness for monocytes (Ouchi, et al., Circulation. 102:1296-1301(2000)). The protein suppresses differentiation of myeloid progenitor cells and has discrete effects on two monocyte cell lines (Yokota, Blood. 96:1723-1732 (2000)). Adiponectin reduces the viability of these cells and blocks LPS induced production of TNF-α. It appears to utilize the ClqRp receptor on normal macrophages and blocks their ability to phagocytose particles (Yokota 2000). Intact or cleaved forms of adiponectin cause increased fatty acid oxidation by muscle cells in treated mice (Fruebis 2001). The protein may also induce metabolic changes in hepatocytes (Yamauchi, et al., 2001; Berg, et al. 2001). Furthermore, adiponectin was found to block myelopoiesis in clonal assays of hematopoietic cell precursors (Yokota 2000). The examples demonstrate that recombinant adiponectin blocks fat cell formation in complex long-term bone marrow cultures (LTBMC). This response appears to result from the induction of cyclooxygenase (COX)-2 and prostaglandins (PGs) in pre-adipocytes.

Macrophages play a central role in immune responses by means of secretion of inflammatory cytokines, phagocytic activity, and antigen presentation. The results show that adiponectin inhibits phagocytosis and LPS-induced TNF-α expression of mature macrophages and suggest that adiponectin may have anti-inflammatory effects. These inhibitory effects of adiponectin on macrophage functions are not due to killing of the cells because the viability of mature macrophages did not change. The mechanisms by which adiponectin cancels TNF-α production and TNF-α gene expression in macrophages stimulated with LPS remain unclear. The kinetic studies indicate that it is unlikely that adiponectin directly neutralizes LPS or blocks LPS receptors. IL-1β and IL-6 gene expression induced by LPS was not affected by treatment with adiponectin, suggesting that signals to macrophages from adiponectin receptors attenuate the TNF-α gene transcription triggered by LPS stimulation. Several cytokines have been found to repress TNF-α synthesis in macrophages, IL-4 and IL-10 suppress inflammatory responses that can inhibit TNF-α synthesis in LPS-stimulated human macrophages. In contrast to adiponectin, IL-4 and IL-10 also inhibit synthesis of IL-1 and IL-6. TGF-β is known to inhibit proinflammatory cytokine production in macrophages, but its inhibition of TNF-α secretion occurs after transcription. Thus, adiponectin is likely to be a unique suppressor of inflammatory responses because of its specific inhibition of TNF-α transcription.

Among the physiologic substances associated with inflammation, E-type prostaglandins (PGE) were shown to inhibit colony formation from CFU-GM and CFU-M but not that from BFU-E. Furthermore, PGE₂ was reported to inhibit TNF-α production but not IL-1α or IL-2β. Target cells and functions of adiponectin are similar to those of PGE and it is now clear that adiponectin can induce PGE synthesis via upregulation of COX2 in at least one cell type (see below). Therefore, adiponectin can influence hermatopoiesis, adipogenesis and immune responses by means of mechanisms involving PGE.

Based on the data showing that adiponectin inhibits production of adipocytes, adiponectin is useful to effect weight loss and as an antiinflammatory. Adiponectin can be administered as the entire 244 amino acid protein, or as fragments thereof retaining the activity as demonstrated in the assays described herein. Based on the functional analysis, it is expected that each subunit will be effective, as well as in the form of a trimer. Fragments including functional domains should also be useful. Conservative substitutions of amino acids may also be made without significantly changing the biological activity. As used herein a conservative substitution refers to the substitution of one amino acid for another having similar size and/or charge.

B. Carriers/Routes/Means for Administration:

Drugs can be administered parenterally or enterally. In the preferred embodiment, drugs are administered orally, in an enteric carrier if necessary to protect the drug during passage through the stomach. Alternative methods of delivery include intravenous, intraperiotoneal, pulmonary, nasal, transbuccal or other trans-membrane delivery, and controlled release formulations.

The adiponectin may be “associated” in any physical form with a particulate material, for example, adsorbed or absorbed, adhered to or dispersed or suspended in such matter, which may take the form of discrete particles or microparticles in any medicinal preparation, and/or suspended or dissolved in a carrier such as an ointment, gel, paste, lotion, or spray.

The adiponectin will usually be administered in combination with a pharmaceutically acceptable carrier. Pharmaceutical carriers are known to those skilled in the art. The appropriate carrier will typically be selected based on the mode of administration. Pharmaceutical compositions may also include one or more active ingredients such as antimicrobial agents, antiinflammatory agents, and analgesics.

Preparations for parenteral administration or administration by injection include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Preferred parenteral vehicles include sodium chloride solution, Ringer's dextrose, dextrose and sodium chloride, lactated Ringer's, or fixed oils. Intravenous vehicles include fluid and nutrient replenishers, and electrolyte replenishers (such as those based on Ringer's dextrose).

Formulations for topical (including application to a mucosal surface, including the mouth, pulmonary, nasal, vaginal or rectal) administration may include ointments, lotions, creams, gels, drops, suppositories, sprays, liquids and powders. Formulations for these applications are known. For example, a number of pulmonary formulations have been developed, typically using spray drying to formulate a powder having particles with an aerodynanmic diameter of between one and three microns, consisting of drug or drug in combination with polymer and/or surfactant.

Compositions for oral administration include powders or granules, suspensions or solutions in water or non-aqueous media, capsules, sachets, or tablets. Thickeners, flavorings, diluents, emulsifiers, dispersing aids or binders may be desirable.

Peptides as described herein can also be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic ac id, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Many formulations for controlled or sustained release are known and commercially available. These are typically formed of a biodegradable polymeric material or polymeric material which is fabricated to provide slow release of the drug. The controlled release composition is preferably a microparticle formulation. The microparticles preferably include a biodegradable, biocompatible polymer such as polylactide that degrades by hydrolysis. In addition to microparticle systems, other controlled-release injectable or implantable formulations can be used. Both degradable and non-degradable excipients can be used in the formulation of injectable or implantable controlled-release formulations, although degradable excipients are preferred. As used herein, the term “microparticles” includes microspheres and microcapsules. The microparticles preferably are biodegradable and biocompatible, and optionally are capable of biodegrading at a controlled rate for delivery of a compound. The particles can be made of a variety of polymeric and non-polymeric materials.

The microparticles can include any biocompatible, and preferably biodegradable polymer, copolymer, or blend. Suitable polymers include polyhydroxy acids, polyorthoesters, polylactones, polycarbonates, polyphosphazenes, polysaccharides, proteins, polyanhydrides, copolymers thereof and blends thereof. Suitable poly(hydroxy acids) include polyglycolic acid (PGA), polylactic acid (PLA), and copolymers thereof. Preferably, the microparticles include poly(D,L-lactic acid) and/or poly(D,L-lactic-co-glycolic acid) (“PLGA”). The preferred material is polylactide.

Microparticles may be prepared using single and double emulsion solvent evaporation, spray drying, solvent extraction, solvent evaporation, phase separation, simple and complex coacervation, interfacial polymerization, and other methods well known to those of ordinary skill in the art. Methods developed for making microspheres for drug delivery are described in the literature, for example, as described in Doubrow, M., Ed., “Microcapsules and Nanoparticles in Medicine and Pharmacy,” CRC Press, Boca Raton, 1992. See also, U.S. Pat. Nos. 5,407,609 to Tice et al., and 5,654,008, to Herbert et al., for methods of making microspheres.

In addition to microparticle systems, other controlled-release injectable or implantable formulations suitable for delivering a compound which induces pseudopregnancy can be used. Both degradable and non-degradable excipients can be used in the formulation of injectable or implantable controlled-release formulations, although degradable excipients are preferred.

Examples of injectable formulations include typical depot formulations prepared with oily and waxy excipients (e.g. similar to Depot Provera™) and in situ gelling systems such as those prepared using sucrose acetate isobutyrate or biodegradable polymers. Examples of implantable formulations include compressed tablet formulations such as those used for controlled release of growth promoters in cattle (e.g. Synovex™), and Compudose™0 (a silicone rubber core coated with a thin layer of medicated silicone rubber containing estradiol). In one embodiment, biodegradable gels and/or implants can be used.

Suitable formulations can be developed by those skilled in the art using any of the approaches described above and typical pharmaceutical excipients.

C. Dosages:

The adiponectin is administered in an amount effective to regulate the size and/or growth of adipocytes or tissue associated therewith. The effective amount will be typically an amount effective to limit adipocyte fat content or adipocyte viability or cell formation or proliferation or to decrease adipose tissue. Compositions as used herein contain an effective amount of adiponectin to treat a patient to achieve the desired regulation in the substantial absence of systemic toxicity.

II. Methods of Treatment

A. Proposed Treatment Schedules

The adiponectin inhibitor is administered in an amount and time period which results in a decrease in the fat content and or number of adipocytes. The latter may be decreased by apoptosis, decreased differentiation from less differentiated cells, and/or decreased proliferation. In the preferred embodiment for the treatment of obesity, patients will receive drug once daily in a dosage effective to decrease the weight to maintenance levels.

B. Types of Patients

The method of treatment should be applicable to both normal overweight individuals and individuals with genetic defects. The method should also be useful in most cases involving weight gains due to hormonal or metabolic defects or drug side effects. In addition to promoting loss of body fat while maintaining lean body mass and being able to sustain weight loss during chronic administration, other benefits of the treatment include normalization of blood glucose levels in obesity related diabetes, and may also be used to reduce appetite (i.e., as an anorexic agent).

The present invention will be farther understood by reference to the following non-limiting examples.

### EXAMPLES

### Example 1

### Adiponectin Inhibits Fat Cell Formation in LTBMC

Methods and Materials

Production and Characterization of Recombinant Adiponectin.

Human recombinant adiponectin was prepared as described by Arita, et al., 1999. Briefly, a 693-bp adiponectin cDNA encoding a peptide leader deficient protein was subdloned into the pET3c expression vector and used to transform host E. coli, BL21(DE3)pLysS. Synthesis of recombinant adiponectin was induced by isopropylthio-β-D-galactoside. Bacterial cells were pelleted and suspended in 50 mM Tris-HCl (pH 8.0) for 1 hour and Triton X-100 at the final concentration at 0.2% and sonicated. The suspended buffer was centrifuged and the pellet was then washed with the same solution. The pellet was precipitated and solubilized with 100 mM Tris-HCl (pH 8.0) containing 7 M guanidine HCl and 1% β-mercaptoethanol. The solubilized protein was refolded in the presence of 200 volumes of 2 M urea, 20 mM Tris-HCI (pH 8.0) for 3 days at 4° C. The refolded protein was concentrated by centrifugal filtration, dialized with 20 mM Tris-HCl (pH 8.0), and purified by DEAE-5PW ion-exchange high performance liquid chromatography (Toso, Japan) equilibrated in 20 mM Tris-HCl (pH 7.2) using a linear gradient of NaCl (0-1 M). SDS-PAGE and Western blotting with adiponectin-specific monoclonal antibodies were used to confirm adiponectin purity. The distribution of its multimetric forms and their formula weights were examined by gel filtration chromatography using Superdex 200 HR 10/30 column (Amersham Pharmacia Biotech., Piscataway, N.J.). Recombinant glutathione S-transferase (GST) was also prepared from E.coli and used as a control. The proteins were dialized with PBS and used at a concentration of 10 μg/ml in culture. After the cell sonication step, all procedures were performed in endotoxin-free buffers and final endotoxin concentrations were less than 0.07 EU/ml checled by Limulus Amebocyte Lysate Pyrogent Plus (BioWhittaker, Walkersiille, Md.).

Reagents

Human insulin was purchased from Roche Diagriosis (Mannheim, Germany). MIBX was purchased from Sigma (St. Louis, Mo.). PGE₂ and Dup-697, purchased from Cayman Chemical (Ann Arbor, Mich.), were used at 1×10⁻⁶ M concentrations.

Tissue, Cells and Mice

Normal human bone marrow was collected by biopsy from the posterior iliac crest of healthy young volunteers with informed consent, and used for immunohistchemical analysis of adiponectin. BMS2 and 3T3-L1 cells were maintained in D-MEM (high glucose) supplemented with 10% fetal calf serum (FCS) (HyClone, Logan, Utah). MS5 cells were maintained in α-MEM medium supplemented with 10% FCS. Balb/c mice at 3-6 weeks old: were obtained from Charles Rivers Breeding Labatories (Wilmington, Me.). B6,129S-^{Ptgs2tm1Jed} (COX-2^{+/-) mice and C}57BL/6 mice (3-5 weeks old) were purchased from the Jackson Laboratory (Bar Harbor, Me.). High mortality and unavailability precluded use of homozygous COX^{-/-} animals in these experiments, but a single targeted allele abrogated preadipocyte responses to adiponectin.

Adiponectin Expression in Bone Marrow.

Expression of adiponectin protein was examined in normal human bone marrow specimens by indirect immunofluorescence methods using the 9108 monoclonal antibody. RT-PCR was used to detect adiponectin transcripts in cDNA prepared from total human bone marrow RNA (CLONTECH, Palo Alto, Calif.). The oligonucleotide primers were 5′-TGTTGCTGGGAGCTGTTCTACTG-3′ (SEQ ID NO:1) and 5′-ATGTCTCCCTTAGGACCAATAAG-3′ (SEQ ID NO:2) for adiponectin, and 5′-CCATCCTGCGTCTGGACCTG-3′ (SEQ ID NO:3) and 5′-GTAACAGTCCGCCTAGAAGC-3′ (SEQ ID NO:4) for β-actin.

LTBMC

LTBMC that support formation of myeloid cells (Dexter cultures) were initiated and maintained by published methods (Dexter, T. M. and Testa, N. G. Methods Cell Biol. 14:387-405 (1976)). Bone marrow cells of normal Balb/c mice (12×10⁶) were cultured in 25-cm² flasks in 5% CO2 at 33 C. The medium consisted of α-MEM supplemented with 100 nM hydrocortisone and 20% horse serum (HyClone). Cultures were treated with adiponectin or bovine serum albumin (BSA) beginning at culture initiation and thereafter weekly for 6 weeks. In some experiments, adiponectin was omitted from the media after 6 weeks of culture, and maintained for another 6 weeks with medium alone.

RT-PCR

Total RNA was isolated from MS5 or BMS2 cells treated with adiponectin for various periods using TRIzol Reagent (GIBCO-BRL, Grand Island, N.Y.) and suspended in DEPC-treated water. After treating total RNA with DNase (GIBCO-BRL), cDNA was made using random hexamers and moloney murine leukemia virus reverse transcriptase (GIBCO-BRL). For PCR, 10 μl of the RT mixtures described above were added to PCR buffer containing 1.5 mM MgCl₂, 1 U Taq polymerase (Perkin Elmer, Norwalk, Conn.), 2 mM each dNTP, and relevant sense and antisense primers. The DNA in the PCR reaction mixtures was amplified using 25 to 35 cycles of 94 C for 1 min, 55 C for 2 min and 72 C for 3 min. The oligonucleotide primers used for these reactions were 5′-GCAAATCCTTGCTGTTCCAAT-3′ (SEQ ID NO:5) and 5′-GGAGAAGGCTTCCCAGCTTTT-3′ (SEQ ID NO:6) for COX-2, and 5′-CCCAGAGTCATGAGTCGAAGGAG-3′ (SEQ ID NO:7) and 5′-CAGGCGCATGAGTACTTCTCGG-3′ (SEQ ID NO:8) for COX-1. Primers for TNF-α, TGF-β, interferon (IFN)-α/β/γ, and limitin were also prepared and used in this study.

Northern Blot Analysis

Poly(A)⁺mRNA was prepared from the indicated samples using oligo(dT) columns (Ambion Inc, Austin, Tex.). Aliquots oifpoly(A)⁺ mRNA (2 μg) were denatured in formamide and formaldehyde at 65 C and electrophoresed on formaldehyde-containing agarose gels. After capillary transfer to nylon membranes (MSI, Westbourough, Mass.), the RNA was crosslinked by UV exposure. cDNA probes for CCAAT/enhancer binding protein-α(C/EBP-α) and adipocyte P2 (aP2) were obtained from ResGen™ Invitrogen (Huntsville, Ala.) and American Type Culture Collection (Manassas, Va.) respectively. Probes with sizes corresponding to PPAR-γ, COX-1 and COX-2 were prepared as PCR products and all probes were radiolabeled with [α-³²P]dCTP using the random prime labeling system (Redi Prime™ II) purchased from Amersham Pharmacia Biotech.

Enzyme-Immunoassay for PGE₂

Confluent MS5 or BMS2 cells prepared in 24-well plates were incubated in 500 μl of media with or without adiponectin. Supernatants from these cultures were examined for the presence of PGE₂ using an enzyme-immunoassay kit purchased from Cayman Chemical.

Adipocyte Differentiation

Differentiation of BMS2 cells to adipocytes was achieved by treatment with 5 μg/ml insulin and 0.5 mM MIBX for 10 days. Differentiation of MS5 cells to adipocytes was achieved by treatment with 5 μg/ml insulin alone for 15 days. Cultures were treated with adiponectin, PGE₂ or Dup-697 from the time of culture initiation. At the end of this period, cultures were photographed and then stained with Nile red to detect lipid accumulation indicative of adipocyte differentiation. The extent of differentiation was estimated by flow cytometry (FACScan; Becton-Dickinson, San Jose, Calif.).

Adherent Bone Marrow Cell Cultures

Adherent bone marrow cell cultures were established with heterozygous knockout COX-2^{+/-} mice or normal C57BL/6 mice. BM cells were suspended at 2×10⁵ per 6 ml of Dexter culture media and seeded in 25-cm² flasks. This cell concentration gives rise to adherent stromal layers without myeloid cell growth. Cultures were treated with adiponecitin or BSA at the time of culture initiation and weekly thereafter for 6 weeks.

Results

Adult bone marrow, like fetal and neonatal tissues, contains brown fat. Adiponectin was originally discovered as a product of subcutanous white fat, and RT-PCR was used to determine if it is also expressed in adult bone marrow. The adiponectin specific primers yielded an amplification product from normal adult marrow cDNA. The specificity of amplification was confirmed by sequencing of the PCR product. An adiponectin specific monoclonal antibody was also used to determine if the protein is present in human bone marrow. Specific staining was found associated with the abundant fat cells in that tissue.

Monomeric recombinant adiponectin has an apparent molecular mass of 32 kD. Additional 64 kD and faint 96 kD bands on SDS-PAGE under non-reducing conditions were also observed, corresponding to dimers and trimers of adiponectin, respectively. No bands were detected above the 102 kD marker. The 64 kD and 96 kD bands disappeared under reducing conditions and only the 32 ID band remained. Adiponectin specific monoclonal antibodies recognized all bands in both conditions by Western-blotting. Although multimeric structures larger than trimers were not detected by SDS-PAGE, gel filtration chromatography showed a wide distribution of recombinant adiponectin with formula weights exceeding trimers. This multimeric character was consistent with native adiponectin in human plasma (Arita 1999), as well as native or recombinant ACRP30, the murine homolog of adiponectin (Scherer, et al. J. Biol. Chem. 270:26746-26749 (1995); Fruebis, et al. Proc. Natl. Acad. Sci. USA. 98:2005-2010 (2001)).

To determine whether adiponectin influenced blood cell formation, LTBMC was established in the presence and absence of this factor. Conditions that favor myeloid cell production were selected, where adipocytes are typically conspicuous in the adherent layer. While no influence on myelopoiesis was found, inclusion of adiponectin in the medium completely inhibited fat cell formation. The negative influence of this protein was reversible and normal numbers of adipocytes were generated when the protein was removed. Additional studies were conducted to determine what cell types were influenced by adiponectin and explore potential regulatory mechanisms.

Bone marrow cultures represent a complex mixture of hematopoietic cells that mature through interactions with an adherent stromal layer composed of fibroblasts, adipocytes, macrophages and endothelial cells. Experiments with three preadipocyte cell lines suggested that preadipocytes could be one target of adiponectin in the bone marrow cultures. The 3T3-L1 cell line rapidly generated adipocytes when insulin was added as an adipogenesis-inducing agent and this response was only slightly inhibited by adiponectin. However, substantial suppression was found with MS5 and BMS2 clones (see below). These experiments demonstrate that preadipocytes can be a direct target of this fat cell product.

### Example 2

### Adiponectin Induces Cyclooxygenase-2 and PGE₂ Synthesis.

TNF-α, TGF-β, interferons and PGE₂ are fat cell products previously shown to inhibit fat cell formation. Thus, their induction was screened for in adiponectin-treated preadipocytes by RT-PCR analysis. Transcripts corresponding to TNF-α or interferon-β were not detectable in MS5 cells even when adiponectin was added to the cultures. Basal expression of TGF-β, interferon-α/β/γ, and a new interferon-like cytokine designated limitin was detectable by RT-PCR, but not obviously influenced by adiponectin. In contrast, it was consistently found that transcripts for COX-2, but not COX-1, were upregulated by adiponectin treatment of either MS5 or BMS2 stromal cell clones. These observations were confirmed by Northern-blot analysis. PGE₂ is known to inhibit adipogenesis and is a substance that depends on COX-2 for its production. Therefore, BMS2 or MS5 cells were allowed to come to confluence before addition of either adiponectin or BSA. PGE₂ concentrations in the supernatants of these cultures were evaluated by ELISA at the indicated times. Adiponectin consistently caused approximately two-fold increases in PGE₂ secretion. Thus, prostaglandin synthesis represents a potential mechanism for inhibition of adipogenesis by adiponectin.

Responses of Pre-Adipocytes to Adiponectin Require COX-2.

Two experimental approaches were used to assess the importance of COX-2 for the inhibition of fat cell formation by adiponectin. BMS2 cells were cultured with MIBX and insulin to induce strong fat cell formation and this response was blocked as expected by inclusion of PGE₂ in the medium. Adiponectin also blocked adipogenesis, while a control GST fusion protein had no influence. The inhibition by adiponectin was not observed when the specific COX-2 inhibitor Dup-697 was present. Inclusion of Dup-697 alone had no influence on fat cell formation. While accumulation of visible fat droplets was blocked by either PGE₂ or adiponectin, the combination of insulin and MIBX still caused a morphological change in adherent layers relative to those in cultures with medium alone. Flow cytometry and Nile red staining of the same cultures was therefore used to extend the microscopic analysis. Lipid accumulation induced by insulin and MIBX was completely blocked by either PGE₂ or adiponectin. The response to adiponectin was substantially blocked by inclusion of the COX-2 inhibitor. Adipocyte gene expression analysis confirmed the cell morphology and lipid accumulation findings. C/EBP-α and PPAR-γ, two transcription factors crucial for adipogenesis, were only weakly expressed in BMS2 preadipocytes, but intensely induced by insulin and MIBX. Either PGE₂ or adiponectin strongly inhibited these increases, and Dup-697 again abrogated the induction by adiponectin. The results were very similar with respect to transcripts for the adipocyte-selective fatty-acid-binding protein aP2.

Adherent bone marrow cell cultures were then prepared with wild type or heterozygous knockout COX-2^{+/-} mice under conditions that favored the formation of numerous fat cells. While adiponectin blocked adipogenesis in cultures of normal C57BL/6 bone marrow, there was minimum effect on cells derived from COX-2^{+/-} animals. These results provide strong evidence that adiponectin directly blocks formation of adipocytes from fat cell precursors through a mechanism that requires induction of COX-2.

The data indicate that the COX-2-dependent prostanoid pathway is important for the suppressive activity of adiponectin on fat cell formation. The response of preadipocytes from COX-2^{+/-} mice to adiponectin was negligible. Poor viability of homozygous COX-2^{+/-} mice precluded their use in the experiments and adiponectin unresponsiveness of the heterozygotes suggests a substantial gene dose effect. Furthermore, a COX-2 inhibitory compound blocked the inhibition of fat cell formation in cultures of cloned preadipocytes. COX-2 is induced in response to pro-inflammatory cytokines or hormones, and is a rate-limiting enzyme in the biosynthesis of PGs. It mediates the conversion of arachidonic acid into PGH_{2,} which is subsequently converted to various kinds of PGs by specific synthases. PGs appear to contribute to fat cell formation in complex ways. For example, PGE₂ and prostacyclin (PGI₂) the two major PGs synthesized by fat cells, appear to have opposing actions on adipogenesis. PGE₂ was shown to negatively regulate fat cell development by reducing cAMP production. Conversely, PGI₂ is proposed as an adipogenic agonist. The data confirm the inhibitory effect of PGE₂ on marrow fat cell differentiation, and further indicate an important contribution to the inhibitory influence adiponectin has on adipogenesis. Other PGs that influence fat cell development include PGJ₂, an important ligand for the adipogenic transcription factor PPAR-γ. This prostaglandin promotes adipocyte differentiation. In contrast, PGF₂ inhibits the adipogenic differentiation of 3T3-L1 cells. Again, PGs with opposing actions are synthesized from PGH₂, a COX-2 product. The 3T3-L1 line generated fat cells in standard culture medium where insulin was the only inducing agent, and this differentiation was minimally affected by addition of either adiponectin or PGE₂. Comparison of 3T3-L1 cells to adiponectin sensitive preadipocytes should be informative about inducible genes and could reveal functional heterogeneity among fat cells in normal tissues.

Two other adipocyte products, agouti and angiotensin II (AGT II) are known to positively contribute to obesity. Agouti induces fatty acid and triglyceride synthesis in cultured adipocytes in a calcium-influx dependent manner. AGT II expression is nutritionally regulated, increasing with high fat diet and fatty acids concomitant with fat mass volume. Adiponectin expression is also affected by diet, but the direction is contrary to that of AGT II (Yamauchi, et al. 2001). AGT II promotes adipocyte differentiation by stimulating release of PGI₂ from mature adipocytes. Thus, PG synthesis appears to play an indispensable role in paracrine actions of adipocyte products on fat cell differentiation.

Modifications and variations will be apparent to those skilled in the art from the foregoing detailed description and are intended to come within the scope of the following claims.

We claim:

1. A method for decreasing fat in adipocytes or the number of adipocytes comprising administering an effective amount of adiponectin to adipocytes or tissue comprising adipocytes.

2. The method of claim 1 wherein the adiponectin is administered to a patient.

3. The method of claim 1 wherein the adiponectin is a fragment of adiponectin.

4. The method of claim 1 wherein the adiponectin reduces appetite.

5. The method of claim 1 wherein the adiponectin is administered in a formulation for enteral delivery.

6. The method of claim 1 wherein the adiponectin is administered in a formulation for parenteral delivery.

7. The method of claim 6 wherein the formulation is for pulmonary delivery.

8. The method of claim 1 wherein the adiponectin is human adiponectin and the adipocytes are human adipocytes.

9. The method of claim 8 wherein the adipocytes are in a patient with diabetes.

10. A pharmaceutical composition comprising adiponectin and a pharmaceutically acceptable carrier for administration of an effective amount of adiponectin to decrease fat in adipocytes or the number of adipocytes.

11. The composition of claim 10 wherein the adiponectin is formulated for enteral administration.

12. The composition of claim 10 wherein the adiponectin is formulated for parenteral administration.

13. The composition of claim 12 wherein the adiponectin is formulated for pulmonary delivery.

14. The composition of claim 10 in a controlled or sustained release formulation.

15. The composition of claim 10 wherein the adiponectin is a fragment.

16. The composition of claim 10 wherein the adiponectin is human adiponectin.

17. A method of making a formulation for decreasing fat in adipocytes or the number of adipocytes comprising adding to a pharmaceutical carrier for parenteral or enteral administration an effective amount of adiponectin to adipocytes or tissue comprising adipocytes.

18. The method of claim 17 wherein the adiponectin is human adiponectin.

19. The method of claim 17 wherein the adiponectin is a fragment of adiponectin.

20. The method of claim 17 comprising making the formulation as a controlled or sustained release formulation.
